(19) United States
(12) Patent Application Publication
(10) Pub. No.: US 2013/0103138 A1
(43) Pub. Date: Apr. 25, 2013

(54) SURFACE MODIFICATION OF MEDICAL DEVICES TO ENHANCE ENDOTHELIAL ADHESION AND COVERAGE

(71) Applicant: Abbott Cardiovascular Systems Inc., Santa Clara, CA (US)

(72) Inventors: Syed Hossainy, Hayward, CA (US); Mikael Trollsas, San Jose, CA (US); Kristen O'Halloran Cardinal, Arroyo Grande, CA (US); Marcus Foley, San Luis Obispo, CA (US); Paul Consigny, San Jose, CA (US)

(73) Assignee: ABBOTT CARDIOVASCULAR SYSTEMS INC., Santa Clara, CA (US)

(21) Appl. No.: 13/655,762

(22) Filed: Oct. 19, 2012

Related U.S. Application Data
(60) Provisional application No. 61/550,222, filed on Oct. 21, 2011.

(51) Int. Cl. A61F 2/82; B05D 5/00

(52) U.S. Cl. 623/1.46; 427/2.25

(57) ABSTRACT

Acceleration of the endothelialization process on implantable medical devices having at least one blood-contacting surface is achieved by a microscale pattern of sub-sections of EC-inductive coatings or EC-conductive coatings and nano/macro textured surfaces. The EC-inductive coating and EC-conductive coating can be applied either on the entire surface of the blood-contacting surface or selective placed on the blood-contacting surface, for example, in particular patterns. In this regard, the EC-conductive and EC-inductive coatings can be selectively placed relative to the textured surface to achieve a desired pattern of texture surface to coatings.

### CROSS REFERENCE TO RELATED APPLICATIONS

The present application claims the benefit under 37 CFR §119(e) of U.S. Provisional Application No. 61/550,222 filed Oct. 21, 2011, the contents of which are incorporated by reference herein in their entirety.

### BACKGROUND OF THE INVENTION

The present invention relates generally to implantable medical devices and, more particularly, to surface texturing and coating which can be placed on an implantable medical device, such as a stent, along with methods for creating such surface textures and coatings that are capable of promoting accelerated and controlled endothelialization with respect to the implantable medical device.

Atherosclerosis is one of the leading causes of death and disability in the world. Atherosclerosis involves the deposition of fatty plaques on the surface of an artery. The deposition of fatty plaques on the artery can cause narrowing of the cross-sectional area of the artery which can lead to blocked blood flow distal to the lesion. When this occurs, ischemic damage to the tissues supplied by the artery can occur. With the advent of percutaneous transluminal coronary angioplasty (PTCA) in the 1970's, catheter techniques originally developed for heart exploration, inflatable balloons were employed to re-open occluded regions in arteries. The procedure was relatively non-invasive, took a very short time compared to by-pass surgery and the recovery time was minimal. However, PTCA brought with it other problems such as vasospasm and elastic recoil of the stretched arterial wall which could undo much of what was accomplished and, in addition, it created a new disease, restenosis, the re-clogging of the treated artery due to neointimal hyperplasia.

The next improvement, advanced in the mid-1980's was the use of a stent to maintain the luminal diameter after PTCA. This for all intents and purposes put an end to vasospasm and elastic recoil but did not entirely resolve the issue of restenosis. That is, prior to the introduction of stents, restenosis occurred in from 30-50% of patients undergoing PTCA. Stenting reduced this to about 15-20%, much improved but still more than desirable.

In the 2000's, drug-eluting stents or DESs were introduced. The drugs initially employed with the DES were cytostatic compounds, that is, compounds that curtailed the proliferation of cells that resulted in restenosis. The occurrence of restenosis was thereby reduced to about 5-7%, a relatively acceptable figure. However, the use of DESs engendered a new problem, late stent thrombosis, the forming of blood clots long after the stent was in place. It was hypothesized that the formation of blood clots was most likely due to delayed healing, a side-effect of the use of cytostatic drugs.

Endothelial cells cover or line the inner surface of the entire vascular system, including the heart, arteries, veins, capillaries and everything in between. Endothelial cells control the passage of materials and the transit of white blood cells into and out of the blood stream. While the larger blood vessels comprise multiple layers of different tissues, the smallest blood vessels consist essentially of endothelial cells and a basal lamina. Endothelial cells have a high capacity to modify or adjust their numbers and arrangement to suit local requirements. Essentially, if it were not for endothelial cells multiplying and remodeling, the network of blood vessel/tissue growth and repair would be impossible.

It has been shown that the body's response to an implanted foreign object is to coat the object with a layer of protein. Macrophages and fibroblasts then encapsulate the object, or cover the object in a layer of collagen. Protein coatings which form on implanted objects in direct contact with blood can be later followed by platelet adhesion and fibrosis. This growth is often referred to as a “pseudo-intima.” Smooth muscle cells (SMCs) and endothelial cells (ECs) may grow over the base coating creating a “neo-intima.” The pseudo-intima, without an endothelial coating, however, is a potentially thrombogenic surface, subject to platelet adhesion, continued fibrotic deposition, and possibly calcification. Platelet adhesion is inhibited or eliminated by a healthy layer of ECs in contact with blood. The creation or formation of a blood-contacting endothelial layer, with or without an underlying layer of SMCs, is highly desired. ECs are known to actively inhibit platelet adhesion, and selectively pass nutrients and cells to and from the underlying tissues. For these reasons, it may be desirable to promote and accelerate the growth of an endothelial layer once the medical device has been implanted within a patient. Because endothelial cells possess certain intrinsic characteristics such as cell regulatory molecules that decrease the incidence of thrombosis or restenosis, stimulating the development of an endothelial cell monolayer on the surface of stents or synthetic grafts may prevent both restenosis and the formation of thrombosis.

The parameters that can induce accelerated endothelialization as investigated in tissue engineering can be basically subclassed into 3 general categories:

A. Chemical surface modification: Biopolymers such as Collagen, Elastin, Silk Elastin, Laminin coatings, RGD with or without non fouling spacers, Hyaluronic acid, Glycosaminoglycan, Endothelial progenitor cell (EPC) capturing antibody can be placed on the surface of the medical implant.

B. Surface Modification: The texture on the surface or bulk porosity in the implant can be modified to enhance endothelialization. For example, the implant can have 30-100 μm porosity. The size and shape distribution on the surface of the implant will result in varying degrees of endothelial growth.

C. Bioactive delivery: Nitric Oxide (“NO”) donor releasing polymers, NO donor released from small, elutible molecules such as c-RGD, pro-endothelialization bioactive components such as platelet-derived growth factor (PDGF) and fibroblast growth factors (FGF) can be associated with the implant.

Accordingly, the parameters to control the amount of endothelialization that can take place include:

1) Composition of the adsorbed protein layer.

2) Physicochemical structure of the adsorbed protein layer. Denatured state, tertiary state, epitope unfolding state.

3) Ratio of Perimeter: surface area, defining the density of pattern per unit area.

4) Relative shape and regional distribution of the pattern on the surface of the medical device.

5) Patterning that can be placed on the surface of the medical device.

6) Texture parameters such as porosity roughness factor.

What is needed is an implantable medical device that includes a surface which promotes accelerated but controlled healing and functionally competent tissue integration on medical implants, such as, but not limited to, bare metal stents, drug eluting stents and absorbable implants. While this would be particularly useful with regard to coronary stents, it would also provide substantial benefit to any manner of implantable medical device. Such a surface may not need a drug or possibly only a small amount of drug application to prevent restenosis. The present invention provides such implantable medical devices and methods for forming such surfaces on implantable medical devices.

### SUMMARY OF THE INVENTION

The present invention addresses these and other problems by providing medical devices that contain at least one blood-contacting surface that is adapted to accelerate endothelialization to control healing and functionally competent tissue integration. This beneficial acceleration of the endothelialization can be achieved by various combinations of microscale pattern of sub-sections of EC-inductive coatings and/or EC-conductive coatings with patterns of nano/macro textured surfaces on the medical device. Medical devices that may benefit from the use of such surface include, but are not limited to, vascular grafts, heart and venous valves, ventricular assist devices, stents, indwelling catheters and filters, pacemaker leads, and plugs for septal defects, aneurysms and heart appendages.

Coating of the medical device with the compositions and methods of the present invention combined with the patterns of textured surfaces that can be generated on the blood-contacting surface of the medical device may stimulate the development of an endothelial cell layer on the surface of the medical device, thereby preventing restenosis as well as other thromboembolic complications that result from implantation of the medical device.

Current clinical science confirms that drug eluting stents are necessary to prevent neointimal hyperplasia. It has been hypothesized that acute injury during implant and chronic injury due to the implant are some of the causes of restenosis. Therefore, a low injury stent which may require less or no drug may be a suitable replacement for a drug eluting stent in the long term. Also, accelerated endothelialization is able to prevent potential late safety outcomes in any blood-contact implant. Functionally competent EC layering may also prevent SMC proliferation and inhibit restenosis.

The present invention is directed to a low injury, implantable medical device, such as a stent, which may be functionally similar to a medical device such as a drug eluting stent. The present invention is capable of creating such an implantable device through the use of selective texturing and/or coating of the surface of the medical device which enhances and accelerates the growth of an endothelial layer once the medical device has been implanted within a patient. The present inventions utilizes macroscale patterns on the implant surface that are based on the following:

a) A pattern consists of two distinct domains including I) EC-inductive or EC-conductive coating and II) EC-inductive or EC-conductive surface texture.

b) The domains I and II can be repeated in a variety of combinatorial ways on the implant surface of the medical device. Using simple mathematics to convey the potential number of ways in which the patterns could be applied, there are N!/(n₁!n₂!) ways that the patterns can be applied to the surface of the implant where N=total domain numbers/length; n₁=coat domain; n₂=texture domain.

c) The surface of the medical implant can be textured first by bead-blasting (sandblasting), physical vapor deposition (PVD), physical abrasion or other deposition techniques known in the art. This will create the precursors of “texture domains.”

d) The EC-inductive and EC-conductive coatings described above can then be applied in a pre-programmed pattern as ink jet deposition, direct dispensing, rubber-stamping and other application methods known in the art.

e) If a drug eluting stent is to be textured, domain I will be the coating that will be applied to the drug eluting stent.

The EC-conductive coating can be selected from a number of composition including of polylysine, poly arginine, fibrinogen, laminin, glycosaminoglycan-rich biopolymer, hyaluronic acid, collagen, elastin, silk-elastin, elastin pentapeptide, RGD, SIKVAV and YIGSR peptide sequence. The EC-inductive coating can be selected from a number of compositon including vascular endothelial growth factor (VEGF), platelet-derived growth factor (PDGF) and c-RGD.

Variations of the above concept can include utilizing NO donors or catalytic generators or c-RGD as the EC-inductive or EC-conductive coating. Additionally, NO donors or c-RGD also could be applied in a pre-programmed pattern by ink-jet deposition, direct dispensing, rubber-stamping and other application methods.

In this manner, the present invention is directed to numerous combinations of surface textures with coatings that can accelerate endothelialization to control healing and functionally competent tissue integration. In one aspect, a textured surface of the medical device can be created on all or part of the blood-contacting surface of the medical device. The textured surface can be created utilizing a number of mechanical etching techniques, mentioned above, such as bead-blasting (sandblasting) and physical abrasion. Mechanical etching of the surface can also be accomplished by subjecting the surface or the coated surface to plasma or IBAD (ion beam assisted deposition), O3 beam, e-beam, Neutron sputtering, reactive ion etching, sand blasting, b-blasting. Chemical etching is also possible by subjecting the coated or partially coated surface to acid, alkali, oxidative electrolysis environment or salt-leaching technique by incorporating soluble granules of salt, sugar, glycine, etc. in the coating on the surface and then leaching out. In another aspect of the invention, only a portion of the blood-contacting surface has a textured surface. Textured surfaces can also be created utilizing deposition techniques such as physical vapor deposition (PVD). In other aspects of the invention, a textured surface can be grafted onto the blood-contacting surface of the medical device. Again, the textured surface, whether created by mechanical etching or deposition or grafting, can cover all or just a portion of the blood-contacting surface. The EC-inductive and EC-conductive coatings used in accordance with the textured surface of the medical device will create a composite blood-contacting surface which can accelerate endothelialization to control healing and functionally competent tissue integration.

The EC-inductive coating and EC-conductive coating identified above can be applied either on the entire surface of the blood-contacting surface or selective placed on the blood-contacting surface, for example, in particular patterns. In this regard, the EC-conductive and EC-inductive coatings can be selectively placed relative to the textured surface to achieve a desired pattern of texture surface to coatings. This can create a synergistic combination of texture and coating to help increase endothelialization.

The scope of the present invention includes bio-absorbable vessel scaffolds and bio-absorbable metallic scaffolds. The scope also includes polymeric, metallic, ceramic, or other inorganic implantable structures functionally designed for, but not limited to, stents, covered stents, synthetic grafts, drug delivery stents and grafts, soft-tissue scaffolding, hard tissue load-bearing, wound-healing, adhesion prevention, artificial heart valves, artificial hearts, fixtures for connecting prosthetic organs to vascular circulation, venous valves, abdominal aortic aneurysm grafts, inferior venal cava filters, permanent drug infusion catheters, embolic coils, embolic materials for vascular embolization, and vascular sutures.

In another aspect, the present invention is directed to methods for forming such patterns consisting of two distinct domains I) EC-inductive or EC-conductive coating and II) EC-inductive or EC-conductive surface texture on a medical device in order to enhance and accelerate the growth of an endothelial layer once the medical device has been implanted within a patient.

These and other advantages of the present invention will become more apparent from the following detailed description of the invention and accompanying exemplary drawings.

### BRIEF DESCRIPTION OF THE DRAWINGS

FIG. 1A is a perspective view of a conventional stent made in accordance with the present invention.

FIG. 1B is a side elevational view showing the stent of FIG. 1A implanted in a patient's vasculature.

FIGS. 2A-2H are cross-sectional views along line 2-2 of FIG. 1 which show particular embodiments of the texturing and coating of the blood-contacting surface of the stent depicted in FIG. 1.

FIG. 3 is a side-elevational view showing a particular embodiment of a stent graft which can be made in accordance with the present invention.

FIG. 4 shows scanning electron microscope images of the various sandblasted surfaces of the test samples.

FIG. 5 is a chart showing the measured surface energy of the various test samples.

FIG. 6 is a chart showing the endothelial cell coverage for each surface of the test samples.

FIG. 7 is a chart showing cell adhesion expressed as average number of cells per image.

FIG. 8 is a chart showing cell spreading results expressed as mean area per cell.

FIG. 9 is a chart showing the levels of PECAM expression per cell for each test sample.

### DETAILED DESCRIPTION OF THE PREFERRED EMBODIMENTS

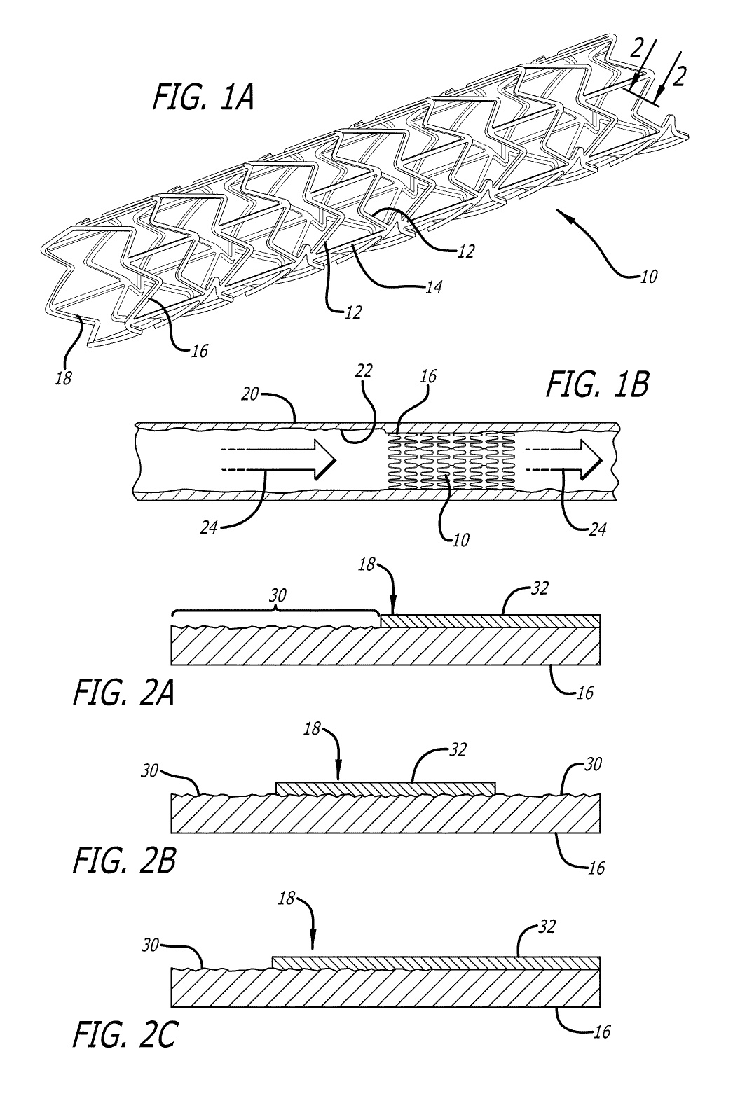

Referring to FIG. 1, an exemplary stent 10 which is made in accordance with the present invention is disclosed. The stent 10 is a patterned tubular device that includes a plurality of radially expanding cylindrical struts 12 disposed generally coaxially and interconnected by connecting struts 14 that are disposed between and connect adjacent cylindrical struts 12. These struts can be any suitable thickness between the stent outer surface 16 and inner surface 18 which makes contact with blood flowing through the stent lumen once the stent 10 is implanted in the vasculature or other body vessel. This inner surface will be herein referred to as the blood-contacting surface 18.

Referring now to FIG. 1B, the stent 10 is shown implanted within a body lumen 20 of the patient. The outer surface 16 of the stent 10 remains in contact with inner surface 22 of the body lumen while the blood-contacting surface 18 in contact with the fluid stream through the body lumen. For example, when the stent 10 is implanted in a blood vessel, the outer surface 16 is in contact with the blood vessel wall, and the blood-contacting surface 18 is in contact with the blood flowing through the vessel (shown by arrows 24).

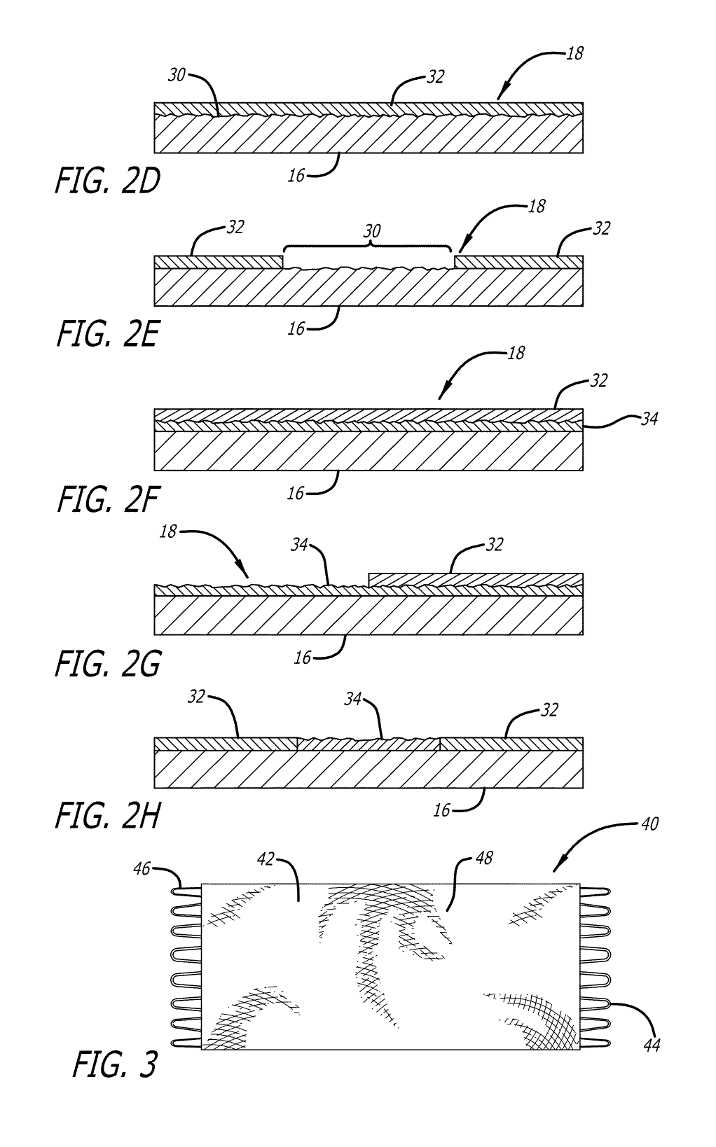

FIGS. 2A-2H show an number of possible combinations of textured surfaces in combination with EC-inductive coatings or EC-conductive coatings. Referring initially to FIG. 2A, one of the possible cross-section of a strut of the stent 10 of FIGS. 1A and 1B is shown. As can be seen in FIG. 2A, the strut includes an outer surface 16 and blood-contacting surface 18 which includes the combination of a surface texture 30 and a coating 32 of an EC-inductive or EC-conductive composition. The texture surface 30 is shown only extending partially across the blood-contacting surface 18 of the stent. In this particular embodiment, the coating 32 is deposited on the untextured portion of the strut directly adjacent to the textured surface 30. This creates a composite blood-contacting surface 18 utilizes the benefits of both a textured surface and coating. The coating 32 could be either an EC-conductive or EC-inductive coating.

Alternatively, the textured surface 30 can extend over the entire blood-contacting surface 18 and the coating 32 can be deposited on a portion of the textured surface 30, as is shown in FIG. 2B, or entirely over the textured surface 30, a is shown in FIG. 2D. This creates unique combination of textured surface to coating which should enhance endothelialization. Also, as is shown in FIG. 2C, the coating 32 could be placed over both the textured surface 30 and untextured surface of the blood-contacting surface 18 so that there is some, but not total, overlap of the coating 32 over the textured surface 30.

FIG. 2E shows the cross-sections of yet other embodiment of the present invention. As can be seen in FIG. 2E, the blood-contacting surface 18 includes a partial textured surface 30. Two domains of coatings 32 are deposited on the blood-contacting surface 18 directly adjacent to the textured surface. In this regard, the coatings 32 can either be an EC-inductive composition, an EC-conductive composition or a combination of the two. In this regard, the composite blood-contacting surface would include a textured surface, an EC-conductive coating and an EC-inductive coating. This shows how many different combinations of coatings and textured surfaces can be created.

The textured surface shown in FIGS. 2A-2D can be created directly on the blood-contacting surface 18 by utilizing mechanical etching techniques, such as, bead-blasting, sandblasting and other mechanical etching techniques. For example, the implant can have 30-100 μm porosity. It should be appreciated that patterns of the textured surface 30 can be created on the blood-contacting surface 18. For example, portions of the blood-contacting surface could be masked accordingly to prevent the masked area from attaining the textured surface. Other etching techniques known in the art could be utilized as well to create textured and non-textured surfaces on the blood-contacting surface to attain the desired patterns of texture. The textured surface can be created utilizing a number of mechanical etching techniques, mentioned above, such as, but limited to, bead-blasting (sandblasting) and physical abrasion. Textured surfaces can also be created utilizing deposition techniques such as physical vapor deposition (PVD). In other aspects of the invention, as is disclosed in FIGS. 2F-2H, a textured surface can be grafted or deposited onto the blood-contacting surface of the medical device. Again, the textured surface, whether created by mechanical etching or deposition or grafting, can cover all or just a portion of the blood-contacting surface.

Mechanical etching of the surface can also be accomplished by subjecting the bare surface or the coated surface to plasma or IBAD (ion beam assisted deposition), O3 beam, e-beam, Neutron sputtering, reactive ion etching, sand blasting, b-blasting. Chemical etching is also possible by subjecting the coated or partially coated surface to acid, alkali, oxidative electrolysis environment or salt-leaching technique by incorporating soluble granules of salt, sugar, glycine, etc. in the coating on the surface and then leaching out.

FIGS. 2F-2H show yet other possibilities of combinations of coatings and textured surfaces. In the these embodiments, the textures surface is not etched directly into the blood-contacting surface 18, but rather, is a layer 34 which is deposited onto the blood-contacting surface 18. Such a deposited layer 34 can also create a textured surface 32 which enhances endothelialization. The deposited material can be grafted onto the blood-contacting surface using techniques known in the art. As can be seen in FIG. 2F, the deposited layer 34 extends over the entire surface of the blood-contacting surface 18. Alternatively, the deposited layer 34 can be placed over only a portion of the blood-contacting surface 18. The coating 32, in turn, can be deposited over the entire deposit layer 34, as is shown in FIG. 2F, or it can be deposited over only a portion of the layer 43, as is shown in FIG. 2G. Alternatively, one or more the coating 32 could be placed adjacent to the deposit layer, as is shown in FIG. 2H. In this regard, the coatings 32 can either be an EC-inductive composition, an EC-conductive composition or a combination of the two.

The EC-inductive and EC-conductive coatings can be applied in a pre-programmed pattern as inkjet deposition, direct dispensing, rubber-stamping and other application methods known in the art. The EC-conductive coating can be selected from a number of composition including of polylysine, poly arginine, fibrinogen, laminin, glycosaminoglycan-rich biopolymer, hyaluronic acid, collagen, elastin, silk-elastin, elastin pentapeptide, RGD, SIKVAV and YIGSR peptide sequence. The EC-inductive coating can be selected from a number of compositon including vascular endothelial growth factor (VEGF), platelet-derived growth factor (PDGF) and c-RGD.

Variations of the above concept can include utilizing NO donors or catalytic generators or c-RGD as the EC-inductive or EC-conductive coating. Additionally, NO donors or c-RGD also could be applied in a pre-programmed pattern by ink jet deposition, direct dispensing, rubber-stamping and other application methods.

An exemplary stent graft 40 is illustrated in FIG. 3. Grafts are typically placed in a blood vessel to either replace a diseased segment that has been removed or to form a bypass conduit through a damaged segment of the vessel wall, for instance, an aneurysm. The graft 40 has a tubular portion 42, which spans the site of the damaged tissue and through which the blood flows. The graft has stent sections, 44 and 46, at both ends that are used to secure the graft to the inside the body vessel. The graft 40 has an outer surface 48, portions of which are in contact with inner surface of the blood vessel wall, and an inner blood-contacting surface (not shown) in contact with the blood which flows through the body vessel. A pattern of the EC-conductive or EC-inductive coating could be placed on a textured inner surface of the tubular portion 42 to enhance the endothelialization process.

It should be appreciated that medical devices other than stents and stent grafts can utilize the benefits of the present invention. For example, the present invention can be implemented with artificial heart valves, artificial hearts, fixtures for connecting prosthetic organs to vascular circulation, venous valves, abdominal aortic aneurysm grafts, inferior venal cava filters, permanent drug infusion catheters, embolic coils, embolic materials for vascular embolization and vascular sutures. In this regard, the various combinations of EC-inductive coating and EC-conductive coating identified above can be applied either on the entire surface of the blood-contacting surface or selective placed on the blood-contacting surface, for example, in particular patterns. Thus, the EC-conductive and EC-inductive coatings can be selectively placed relative to the textured surface on the particular medical device to achieve a desired pattern of texture surface to coatings.

### Experimental Sandblasting Testing

Testing was performed to see the effect of surface modifications of a medical implant and to determine the ability of the modified surface to enhance growth of endothelial cells. The size and shape distribution of the surface texturing on the surface of the implant will result in varying degrees of endothelial growth. Cobalt chromium is a widely used metal in cardiovascular and orthopedic applications and was chosen as the sample to be tested. The following procedures were implemented to characterize the surface properties of various cobalt chromium surface modifications and to evaluate the response of endothelial cells to the modified surfaces.

Cobalt chromium samples were sandblasted using various abrasive aluminum oxides at several operating pressures. The resulting surfaces were imaged using a scanning electron microscope (SEM). Results will show the feature size of each surface, quantified in ImageJ using length and area measurements. The surface energy of each modification was determined using contact angle measurements. Results will also show effects on endothelial cell coverage, evaluated through morphometric analysis of an en face fluorescent stain for platelet-endothelial cell adhesion molecule (PECAM-1) and the average levels of PECAM-1 expressed per cell. In a separate analysis, a cell adhesion assay was performed to quantify differences in initial cell adhesion to each surface.

### Methods for Sandblasting

L-605 cobalt chromium alloy (High Temp Metals, 12910 San Fernando Road, Sylmar, Calif.) with a thickness of 0.010″ was cut into 0.75″ square samples in preparation for sandblasting. Each sample was sandblasted using a 0.5 mm nozzle handheld sandblaster (TCP Global, 6695 Rasha Street, San Diego, Calif.). 220 mesh, 400 mesh and 800 mesh aluminum oxide abrasive media was used in the sandblaster (Kramer Industries, 140 Ethel Road West, Piscataway, N.J.) to achieve different surface finishes. The sandblaster operating pressure was varied from 20 psi static to 60 psi static and the samples were sandblasted until a uniform surface finish was observed. FIG. 1 shows the sandblasting system used during testing which includes a 2 gallon air compressor and a handheld airbrush.

### Feature Size Characterization

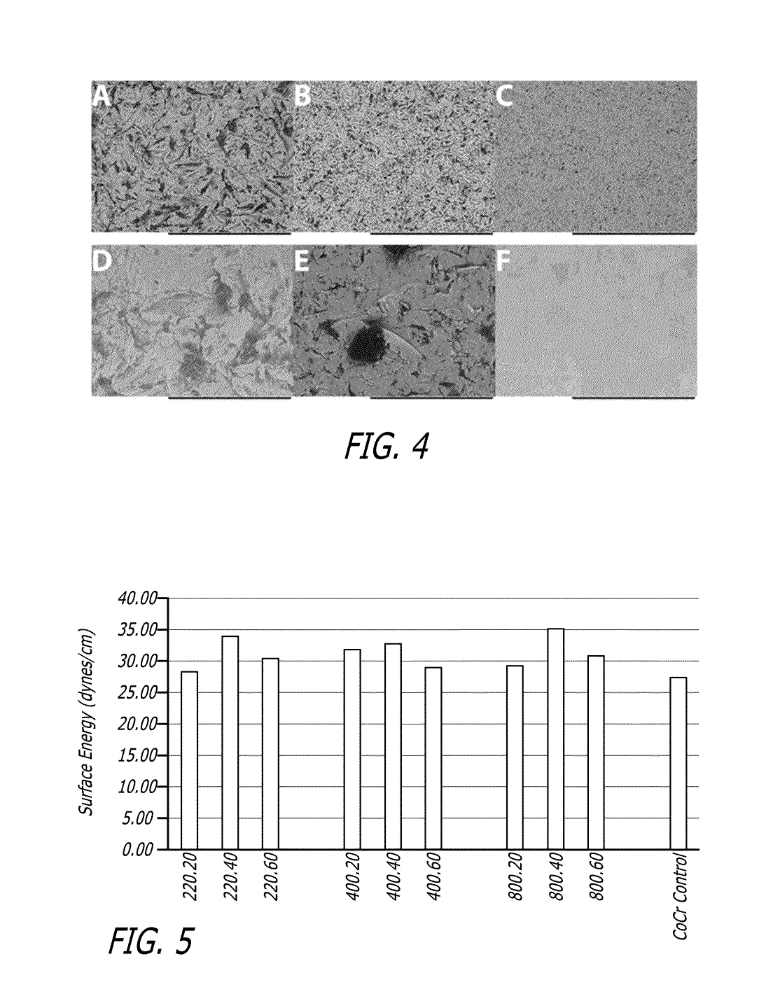

FIG. 4 shows scanning electron microscope images of several sandblasted surfaces of test samples. SEM images were taken at magnifications of 1000× and 4000×. The feature size was quantified using the trace tool in ImageJ, and a grid to prevent measurement bias. \

### Surface Energy

The surface energy of each surface modification was characterized using contact angle measurements. Four liquids were used in the contact angle tests: DI water, methyl salicylate, diethylene glycol, and 70% ethanol. After measuring the contact angle of each drop, a Zisman plot was created to calculate the surface energy. A chart showing the measured surface energy is disclosed in FIG. 5 and discussed further below.

### Cell Culture

HUVECs (human umbilical vein endothelial cells) were cultured on each sample for 48 hours, with an initial density of 60,000 cells/cm2. The cells were then fixed in 10% formalin in preparation for immunostaining

### Immunostaining for PECAM-1

Cells were incubated in a primary antibody against PECAM-1 (platelet endothelial cell adhesion molecule 1) (CD31, Invitrogen Cat. No. 37-0700, Carlsbad, Calif.) followed by an incubation in a fluorescent secondary antibody (Alexa Fluor 488 goat anti-mouse, Invitrogen Cat. No. A-11001, Carlsbad, Calif.). Cell nuclei were counterstained using Hoechst 33258 (Invitrogen Cat. No. H3569, Carlsbad, Calif.).

Cells were incubated in a primary antibody against PECAM-1 (platelet endothelial cell adhesion molecule) followed by an incubation in an Alexa Fluor 488 fluorescent secondary antibody. Fluorescent images were acquired using a wide field fluorescent microscope. The levels of PECAM-1 expression were calculated by mean cell intensity using ImageJ and plotted in Excel. Endothelial cell coverage based on a positive reaction to the PECAM-1 stain was visually semi-quantified for each surface and expressed as a mean percentage of total surface area.

### Sandblasting

Scanning electron microscope images of the sandblasted surfaces showed uniform feature coverage as expected. FIG. 4 shows selected images of these surfaces. The images clearly show feature size decreases with the grain size of the abrasive media. This will be quantified in the next section.

### Feature Size Characterization

The results of the feature size characterization performed on the SEM images show the variability produced by the various aluminum oxides (see Table 1 below). Subcellular features (less than 10 μm in length) were achieved using 400 mesh and 800 mesh aluminum oxides.

TABLE 1
Results of feature size characterization for each mesh size and pressure.

| | 220 mesh | | 400 mesh | | 800 mesh | |
|---|---|---|---|---|---|---|
| | Averagelength | Average area | Average length | Average area | Average length | Average area |
| | (μm) | (μm²) | (μm) | (μm²) | (μm) | (μm²) |
| 20 PSI | 8.6 | 17.5 | 3.7 | 3.2 | 1.3 | 0.4 |
| 40 PSI | 10.4 | 24.5 | 3.9 | 7.3 | 1.6 | 1.1 |
| 60 PSI | 11 | 25.5 | 4.9 | 7.4 | 1.7 | 1.7 |

| 46 mesh | | #8 glass beads | |
|---|---|---|---|
| Average ength | Average area | Average length | Average area |
| (μm) | (μm²) | (μm) | (μm²) |
| 20.4 | 117.7 | 13 | 71.9 |

### Surface Energy

Surface energy was generally higher on the modified surfaces compared to unmodified cobalt chromium, but no clear trend was observed between various media or pressures. FIG. 5 provides a chart depicting the surface energy for the various mesh sizes and pressures used on the cobalt chromium samples. In FIG. 5, the labeled horizontal axis is expressed as xxx.yy where xxx denotes mesh size and yy denotes pressure in PSI.

### Immunostaining for PECAM-1

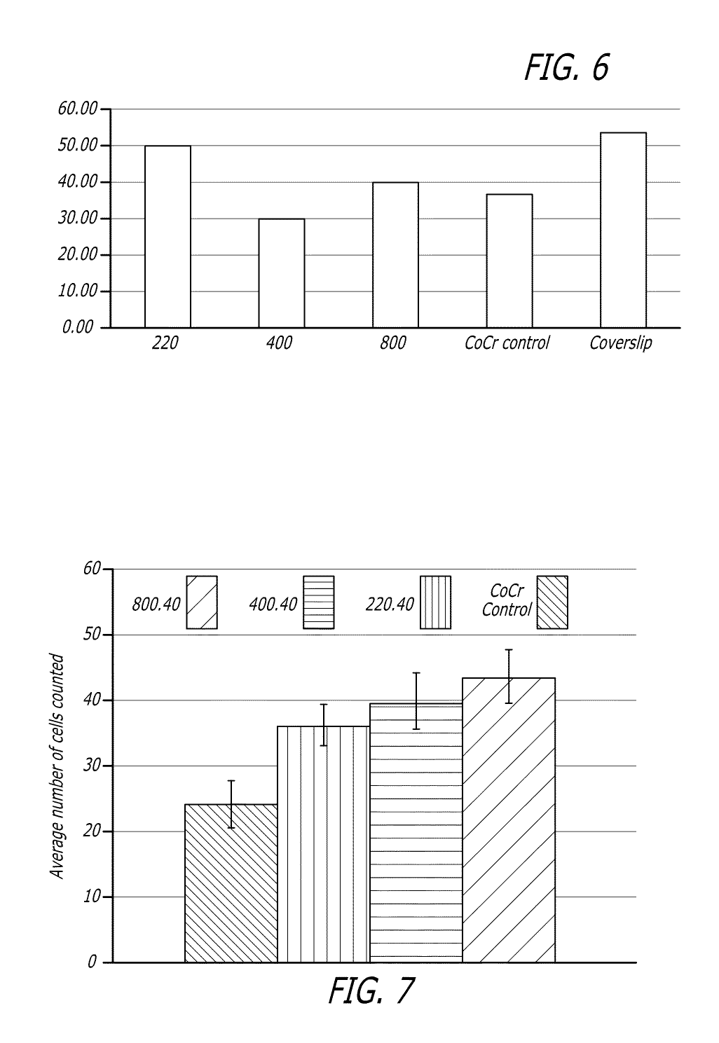

Immunostaining successfully showed PECAM-1 expression in endothelial cells on each surface. The levels of the PECAM-1 expression were calculated by mean cell intensity using ImageJ and were plotted in Excel. Endothelial cell coverage based on positive PECAM-1 staining was visually semi-quantified for each surface of the test samples and expressed as a means percentage of the total surface area. The results showed no statistically significant difference between the surfaces. FIG. 6 shows the endothelial cell coverage for each sample surface. In FIG. 6, the endothelial cell coverage for each surface is expressed as a percentage of total surface area.

Cells were fixed and stained after initial cell seeding using a fluorescent rhodamine phalloidin stain to examine spreading of the cells. The number of cells per image and average cell areas were quantified using ImageJ. A separate study was performed to assess cell adhesion and spreading on samples modified at the 40 psi pressure setting. Cells were fixed and stained 12 minutes after initial cell seeding using a bisbenzimide fluorescent stain to examiner adhesion.

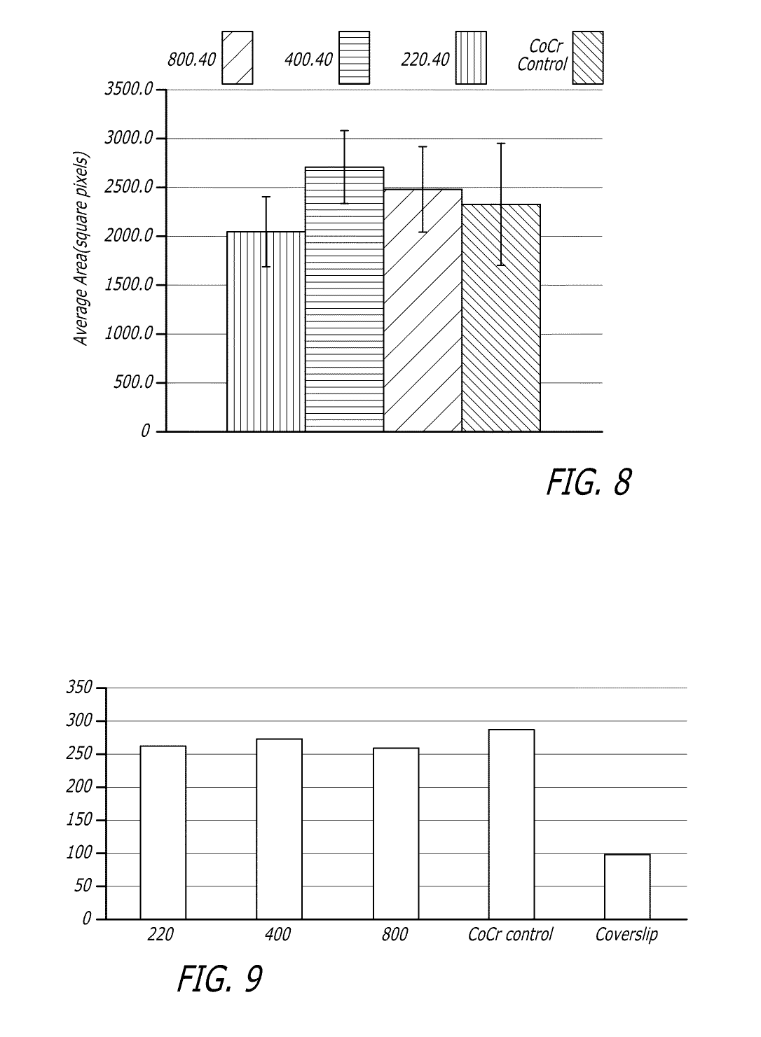

FIG. 7 shows cell adhesion expressed as average number of cells per image for each test sample. The labeled test samples are expressed as xxx.yy where xxx denotes mesh size and yy denotes pressure in PSI used in creating the particular sample. FIG. 8 shows cell spreading results expressed as mean area per cell for each of the test samples. Again, the labeled test samples are expressed as xxx.yy where xxx denotes mesh size and yy denotes pressure in PSI used in creating the particular sample. The levels of PECAM-1 expression per cell were also quantified, and show significant increases on all of the CoCr surfaces when compared to the glass coverslip. The results are shown in FIG. 9. The test samples in FIG. 9 are designated by the size of the mesh size used in creating the test sample.

A relatively linear relationship between grain size and feature size was observed on the sandblasted surfaces. The surface energy and levels of PECAM-1 expression were higher on the modified surfaces than the control surface, but no clear trend was observed between modifications. The modified surfaces showed enhancement of endothelial cell adhesion and spreading without diminishing cell coverage. The benefits, availability and cost effectiveness of sandblasting make it a useful technique to determine the impact of surface texturing on cell-material interactions.

Data obtained during the testing is generally disclosed in FIGS. 14-19 of U.S. Provisional Application No. 61/550,222 filed Oct. 21, 2011, the contents of which are incorporated by reference herein in their entirety.

It is therefore intended that the foregoing detailed description be regarded as illustrative rather than limiting, and that it be understood that it is the following claims, including all equivalents, that are intended to define the spirit and scope of this invention.

We claim:

1. An implantable medical device comprising:
a structural body having a blood-contacting surface, wherein the blood-contacting surface includes a pattern comprising of a first domain and a second domain, the first domain being either an EC-inductive surface texture or an EC-conductive texture and the second domain being either an EC-inductive coating or an EC-conductive coating, the first domain and second domain stimulating adherence and proliferation of endothelial cells on the blood-contacting surface of the structural body to rapidly form a confluent endothelium in vivo.

2. The implantable medical device as recited in claim 1, wherein the EC-conductive coating is selected from the group consisting of polylysine, poly arginine, fibrinogen, laminin, glycosaminoglycan-rich biopolymer, hyaluronic acid, collagen, elastin, silk-elastin, elastin pentapeptide, RGD, YIGSR and SIKVAV peptide sequence

3. The implantable medical device as recited in claim 1, wherein the EC-inductive coating is selected from the group consisting of VEGF, PDGF and c-RGD.

4. The implantable medical device as recited in claim 1, wherein the EC-conductive texture is an etched surface on the blood-contacting surface of the structural body.

5. The implantable medical device as recited in claim 4, wherein only a portion of the blood-contacting surface has an etched surface.

6. The implantable medical device as recited in claim 4, wherein all of the blood-contacting surface has an etched surface.

7. The implantable medical device as recited in claim 1, wherein the EC-inductive texture or EC-conductive texture is a deposited material on the blood-contacting surface of the structural body.

8. The implantable medical device as recited in claim 7, wherein the deposited material covers all of the blood-contacting surface.

9. The implantable medical device as recited in claim 7, wherein the deposited material covers only a portion of the blood-contacting surface.

10. The medical device of claim 1, wherein the medical device is a stent, covered stent, flow diverter, synthetic graft, artificial heart valves, artificial hearts, fixtures for connecting prosthetic organs to vascular circulation; venous valves, abdominal aortic aneurysm grafts, inferior venal caval filters, permanent drug infusion catheters, embolic coils, embolic materials for vascular embolization, or vascular sutures.

11. An implantable medical device comprising:
a structural body having a blood-contacting surface, wherein the blood-contacting surface includes a pattern comprising of a first domain and a second domain, the first domain being a surface texture and the second domain being either an EC-inductive coating or an EC-conductive coating, the first domain and second domain stimulating adherence and proliferation of endothelial cells on the blood-contacting surface of the structural body to rapidly form a confluent endothelium in vivo.

12. The implantable medical device as recited in claim 11, wherein the surface texture covers the entire blood-contacting surface of the structural body.

13. The implantable medical device as recited in claim 11, wherein the surface texture covers a portion of the blood-contacting surface of the structural body and the EC-inductive coating or an EC-conductive coating is placed over the portion of the blood-contacting surface which does not have the surface texture.

14. The implantable medical device as recited in claim 11, wherein the EC-conductive coating is selected from the group consisting of polylysine, poly arginine, fibrinogen, laminin, glycosaminoglycan-rich biopolymer, hyaluronic acid, collagen, elastin, silk-elastin, elastin pentapeptide, RGD, SIKVAV and YIGSR andpeptide sequence.

15. The implantable medical device as recited in claim 11, wherein the EC-inductive coating is selected from the group consisting of VEGF, PDGF and c-RGD.

16. A method for forming an implantable medical device which has a blood-contacting surface which stimulates adherence and proliferation of endothelial cells thereto to rapidly form a confluent endothelium, comprising:
forming a structural body having a blood-contacting surface;
forming a pattern of an EC-inductive surface texture or an EC-conductive texture on the blood-contacting surface; and
applying an EC-inductive coating or an EC-conductive coating on the blood-contacting surface.

17. The method according to claim 16, wherein the EC-conductive coating is selected from the group consisting of polylysine, poly arginine, fibrinogen, laminin, glycosaminoglycan-rich biopolymer, hyaluronic acid, collagen, elastin, silk-elastin, elastin pentapeptide, RGD, SIKVAV and YIGSR peptide sequence

18. The method according to claim 16, wherein the EC-inductive coating is selected from the group consisting of VEGF, PDGF and c-RGD.

19. The method according to claim 16, wherein the forming of the EC-conductive texture is performed by mechanically etching the blood-contacting surface.

20. The method according to claim 19, wherein only a portion of the blood-contacting surface is mechanically etched.

21. The method according to claim 19, wherein all of the blood-contacting surface is mechanically etched.

22. The method according to claim 16, wherein the EC-conductive texture is formed by depositing a material on the blood-contacting surface of the structural body.

23. The method according to claim 16, wherein the deposited material covers all of the blood-contacting surface.

24. The method according to claim 16, wherein the deposited material covers a portion of the blood-contacting surface.

25. The method according to claim 16, wherein the EC-inductive or EC-conductive coatings is applied in a pre-programmed pattern.

26. The method according to claim 16, wherein the EC-inductive or EC-conductive coatings is applied by ink-jet deposition

27. The method according to claim 16, wherein the EC-inductive or EC-conductive coatings is applied by rubber-stamping.

28. The method according to claim 16, wherein the medical device is a stent, covered stent, synthetic graft, artificial heart valves, artificial hearts, fixtures for connecting prosthetic organs to vascular circulation; venous valves, abdominal aortic aneurysm grafts, inferior venal caval filters, permanent drug infusion catheters, embolic coils, embolic materials for vascular embolization, or vascular sutures.

29. A method for forming an implantable medical device which has a blood-contacting surface which stimulates adherence and proliferation of endothelial cells thereto to rapidly form a confluent endothelium, comprising:
forming a structural body having a blood-contacting surface;
forming a pattern of surface texture on the blood-contacting surface; and
applying an EC-inductive coating or an EC-conductive coating on the blood-contacting surface.

29. The method according to claim 28, wherein the pattern of surface texture covers the entire blood-contacting surface of the structural body.

30. The method according to claim 28, wherein the pattern of surface texture covers a portion of the blood-contacting surface of the structural body and the EC-inductive coating or an EC-conductive coating is applied over the portion of the blood-contacting surface which does not have the surface texture.

31. The method according to claim 28, wherein the EC-conductive coating is selected from the group consisting of polylysine, poly arginine, fibrinogen, laminin, glycosaminoglycan-rich biopolymer, hyaluronic acid, collagen, elastin, silk-elastin, elastin pentapeptide, RGD, SIKVAV and YIGSR peptide sequence.

32. The method according to claim 28, wherein the EC-inductive coating is selected from the group consisting of VEGF, PDGF and c-RGD.
